(19) United States
(12) Patent Application Publication
(10) Pub. No.: US 2016/0287299 A1
(43) Pub. Date: Oct. 6, 2016

(54) Bone fixation device and method

(71) Applicant: AUBREY GROUP, INC., Irvine, CA (US)

(72) Inventor: Charles Lang, Oxnard, CA (US)

(21) Appl. No.: 14/415,447

(22) PCT Filed: Jul. 15, 2013
(86) PCT No.: PCT/US13/50480

§ 371 Date: Jan. 16, 2015

Related U.S. Application Data
(60) Provisional application No. 61/671,703, filed on Jul. 14, 2012.

(51) Int. Cl. A61B 17/82; A61B 17/88

(52) U.S. Cl. CPC A61B 17/82; A61B 17/8869; A61B 17/8863

(57) ABSTRACT

A bone fixation device and method for compressing bone or bone fragments temporarily or permanently using a tension strip to surround the exterior surface of the bone or bone fragments and applying a force to the tension strip wherein the bone or bone fragments are compressed together. Bone or bone fragments are able to heal more quickly when compressed under super natural pressure. The temporary tension strips may be made of bioresorbable polymer and a radiopaque dopent so that the tension strip is visible under fluoroscopic observation.

### CROSS REFERENCE TO RELATED APPLICATIONS

U.S. provisional application No. 61/671,703 dated Jul. 14, 2012 and PCT application number PCT/US2013/050480 dated Jul. 15, 2013 the contents of which are hereby incorporated by reference.

### BACKGROUND OF THE INVENTION

1. Field of the Invention

The present invention relates generally to the field of medical devices. In particular, the present invention relates to devices for bone fixation and methods for using the device.

2. Description of the Related Art

External fixation is a surgical treatment used to set bone fractures in which a cast would not allow proper alignment of the fracture. In this kind of reduction, holes are drilled into uninjured areas of bones around the fracture and special bolts or wires are screwed into the holes. Outside the body, a rod or a curved piece of metal with special ball-and-socket joints joins the bolts to make a rigid support. The fracture can be set in the proper anatomical configuration by adjusting the ball-and-socket joints.

Certain difficulties arise with the traditional manner of performing the external fixation; it can be difficult for the surgeon to estimate the necessary amount of tension force correctly. Also, currently available methods of bone fixation are time consuming and often require more than on technician to properly align bone and operate fixation devices simultaneously.

Alternative bone fixation methods include open reduction internal fixation which involves the implementation of implants to guide the healing process of a bone, as well as the open reduction, or setting, of the bone itself. Open reduction refers to open surgery to set bones, as is necessary for some fractures. Internal fixation refers to fixation of screws and plates, intramedullary bone nails (femur, tibia, humerus) to enable or facilitate healing. Rigid fixation prevents sliding motion across lines of fracture to enable healing and prevent infection. Internal fixation techniques are often used in cases involving serious fractures such as comminuted or displaced fractures or in cases where the bone would otherwise not heal correctly with casting or splinting alone.

Risks and complications can include bacterial colonization of the bone, infection, stiffness and loss of range of motion, non-union, mal-union, damage to the muscles, nerve damage and palsy, arthritis, tendonitis, chronic pain associated with plates, screws, and pins, compartment syndrome, deformity, audible popping and snapping, and possible future surgeries to remove the hardware.

### BRIEF SUMMARY OF THE INVENTION

The disclosed embodiments provide a device and method for bone fixation in a patient.

In a particular embodiment, a tension strip is wrapped around bone fragments, the amount of tension required to provide compression to bone or bone fragments is adjusted on the tensioning device. Devices used in the field and related to compressing bone fragments are disclosed in U.S. Pat. No. 4,793,385 issued to Dyer, U.S. Pat. No. 5,250,049 issued to Michael, U.S. Pat. No. 6,076,234 issued to Betts, U.S. Pat. No. 6,589, 246, issued to Hack, U.S. Pat. No. 7,641,677 issued to Weiner, U.S. Pat. No. 7,806,895 issued to Weier and U.S. Pat. No. 8,034,076 issued to Criscuolo. Additional US Patent Applications have published including: 20080199824 Hargadon, 20090171357 Justin, 20100274248 Dell, 20100292698 Hulliger, 20100298828 Chico, 20100298829 Schaller, 201000305571 Pratt, 20110034928 Fernandez, and 20110112537 Bernstein. This and all other referenced patents are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

The present invention achieves its objects by providing a device and method that quickly allows access around the bone fragments and a robust device for applying a preset amount of pressure to the bone.

### OBJECTS OF THE INVENTION

A bone fixation method comprising a tension strip with a low profile head for receiving a tension strip tail, the tension strip tail placed around a bone with a hollow guide and the hollow guide removed, a tension device (having a long straight body) comprising an engagement portion, a handle portion, and a cutting portion, the engagement portion further comprising a first slot and a second slot, wherein the low profile head is engaged with the tension device at the first slot and the tension strip tail is engaged with the tension device at the second slot, wherein the handle portion is actuated to create a force to move the tension strip tail relative to the low profile head creating tension pressure circumferentially around the bone optionally the cutting portion can be set to cut off excess tension strip tail at a preset pressure setting.

A tension device that accommodates various widths and thickness of tension strips.

Tension strips with low profile head and tail portions.

Bone plate system with a metal or polymeric bar with grooves rather than holes for inexpensive manufacturing methods and adapted to receive tension strips in the groove to that the tension strip does not slide along the axial direction of the bar.

Tension strip comprised of biodegradable material (commonly available polymers) that can be doped with radiopaque materials such as iodine compounds or metal strips for ease of visualization post procedure with x-ray or fluoroscopic methods.

Tension strip tail ejection orientation is out of the top of the tension device whereas currently available methods eject cable in the downward direction, often times obstructing technician's access to the wound site.

Displacement distance between the engaging head and tension strip exit port, reduce friction and speeds user's loading and reloading if necessary.

Preset tension on the tensioning device to automatically set for young bones, adult bones and geriatric bones. The tensioning device can be set to cut automatically when the preset tension is reached. Or the device can be set not to cut automatically.

Problems with Prior Art

Current tools for applying tension to fastners have not been ergonomic, often having designs that obscure or make visualization of the fracture difficult. The present invention has an elongate body that is thin so as not to obscure a technician's vision of the fracture.

Current tool designs incorporate vertical handles that can inhibit technician access to surgical sites.

Currently available methods do not incorporate the use of a tension strip guide so that tensioning cannot be performed in short duration sequential order.

The manners in which the invention achieves its objects and other objects which are inherent in the invention will become more readily apparent when reference is made to the accompanying drawings wherein like numbers indicate corresponding parts throughout.

### BRIEF DESCRIPTION OF THE DRAWINGS

FIG. 1 is a cross section view of a bone fracture.

FIG. 2 is a drawing of a hip implant.

FIG. 3 is a perspective view of a hip implant that has fractured a femur and metal strips.

FIG. 4 is a drawing of metal tension strips compressing two bone fragments together.

FIG. 5 is a top view of a preferred embodiment of a tension strip showing a serrated or textured contact surface.

FIG. 6 is a side view of a preferred embodiment of a tension strip showing a serrated or textured contact surface.

FIG. 7 is a perspective cross section of a preferred embodiment of a tension strip engagement head.

FIG. 8 is a cross section view of a preferred embodiment of a tension strip head.

FIG. 9 is a cross section view of a preferred embodiment of a tension strip showing a flat contact surface.

FIG. 10 is a cross section view of a preferred embodiment of a tension strip showing a curved contact surface.

FIG. 11 is a cross section view of a preferred embodiment of a tension strip showing a smooth contact surface.

FIG. 12 is a cross section view of a preferred embodiment of a tension strip showing a serrated or textured contact surface.

FIG. 13 is a side view of a preferred embodiment of a positioning device used for placing tension strips around a bone fragment.

FIG. 14 is a cross sectional view of a positioning device used for placing tension strips around a bone fragment.

FIG. 15 is a side view of a positioning device with designated openings.

FIG. 16 is prior art showing relevant design for commercially available zip tie gun.

FIG. 17 is prior art showing relevant design for commercially available zip tie gun showing an adjustable pre-set tensioning mechanism.

FIG. 18 shows a preferred embodiment of a tensioning device.

FIG. 19 shows a close up view of preferred embodiment of a tensioning device.

### DETAILED DESCRIPTION OF THE INVENTION

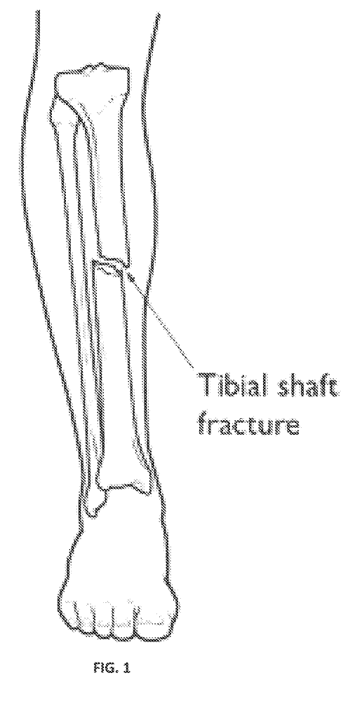

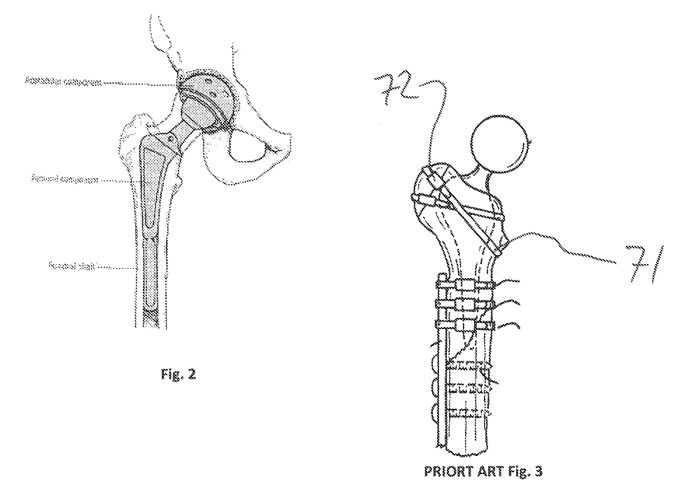

FIG. 1 illustrates a typical bone fracture of a human tibia into two bone fragments. FIG. 2 illustrates a commercially available hip replacement joint. During a typical hip replacement surgery the technician may use a hammer to push a femoral anchor into the femur bone with great force. It is not uncommon with older patients to have hairline fractures or major fractures occur during the anchoring process in an artificial hip replacement. FIG. 3 illustrates how prior art metal bands 71 and fasteners 72 could be used in multiple locations to hold fractured bone intact so that an artificial hip implant can be stabilized and bone can heal.

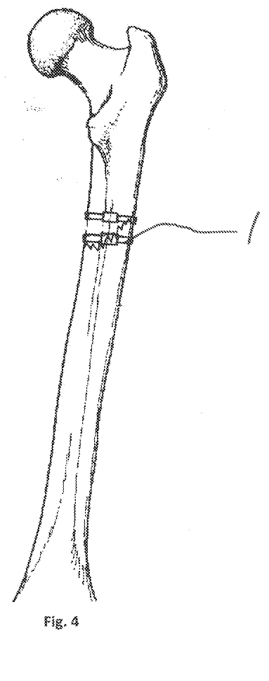

FIG. 4 illustrates a method of compressing the two bone fragments together with tensioning strips 1.

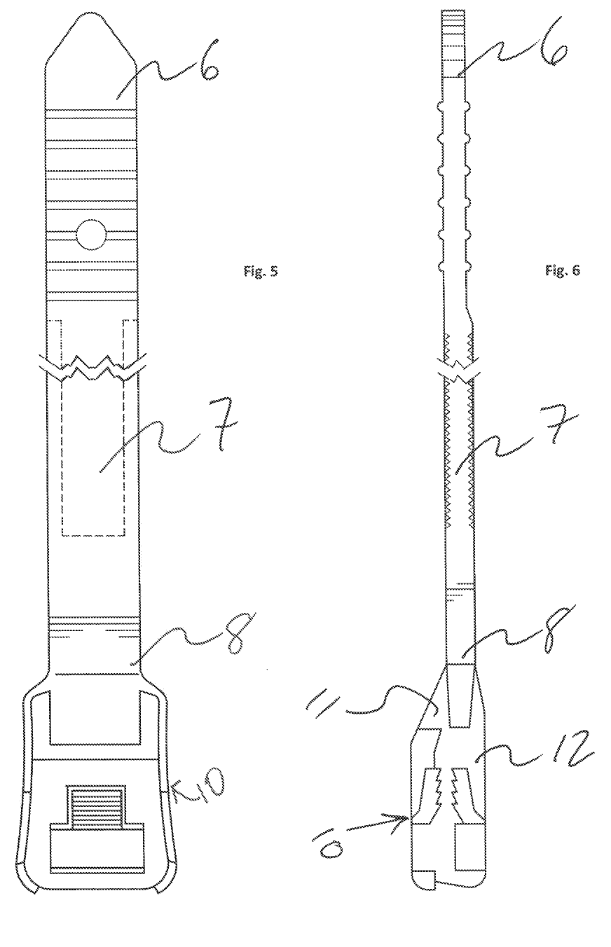

FIGS. 5 and 6 show a tension strip 1 with differentially modified surfaces along the axis. The tension strip 1 comprises a proximal portion 6 that has ribs or texturing for attaching to a tension strip head 10 that further comprises an upper portion 11 and a bottom portion 12. The tension strip head 10 is attached to a tension strip distal portion 8. Tension strip 1 has a medial portion 7 disposed between the proximal portion 6 and distal portion 8. The medial portion 7 is disclosed with various surface modifications to improve friction between the tension strip 1 and bone (see additional FIGS. 9-12). The thickness of the tension strip 1 may also vary as well as the type of texture.

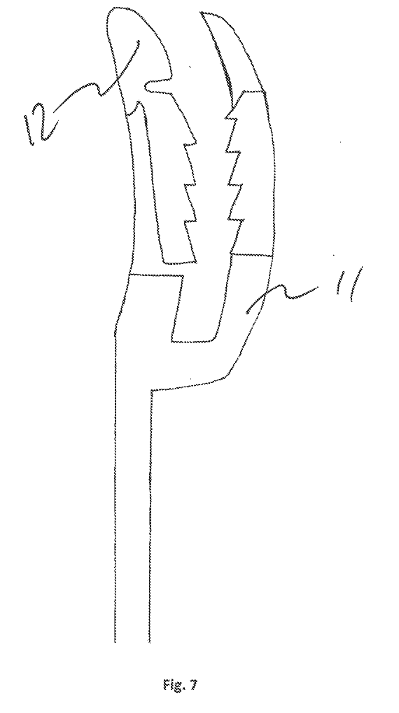

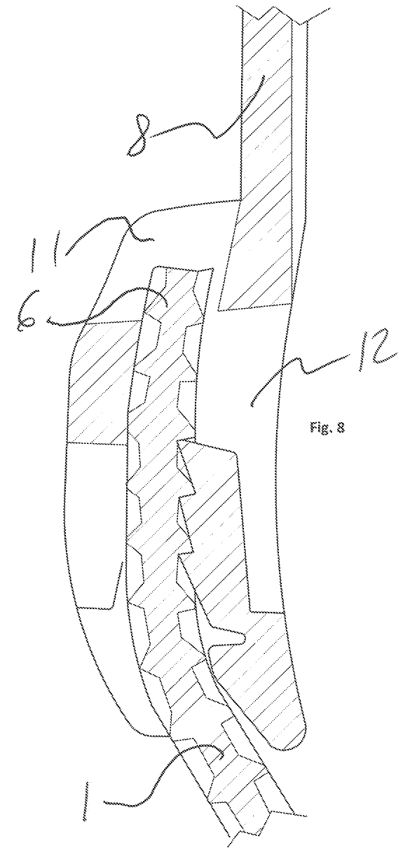

FIG. 7 illustrates a preferred embodiment of a tension strip head 10, often referred to as a ratchet in common commercial uses such as an electrician organizing many loose wires into a clean bundle of wires. The top portion 11 of the head 10 is fixed to the tension strip body distal portion 8. The bottom portion 12 of the head 10 is flexibly attached to the tension strip body distal portion 8. Either or both the top and bottom portions 11,12 of the tension strip head 10 may be textured (such as with ridges) to increase friction and ensure locking with the proximal portion 6 of the tension strip 1. The proximal portion 6 of the tension strip 1 is folded over and passes between the top and bottom portions 11,12 of the tension strip head 10. As the proximal portion 6 of the tension strip 1 continues to pass through the head 10 ribs on the tension strip surface temporarily displace the bottom portion 12 of the tension strip head 10. Once the tension strip rib completely clears the ridge on the bottom portion 12 of the tension strip head 10 the bottom portion 12 of the tension strip head 10 returns to its natural unflexed position. In this unflexed position the rib and ridge of the bottom portion 12 of the tension strip head 10 are in frictional contact. Once the proximal portion 6 of the tension strip has passed through the tension strip head 10 a closed loop is formed and the loop cannot be loosened, it can only be tightened. FIG. 8 shows a close up cross section of a tension strip head 10 with the tension strip proximal portion 6 engaged.

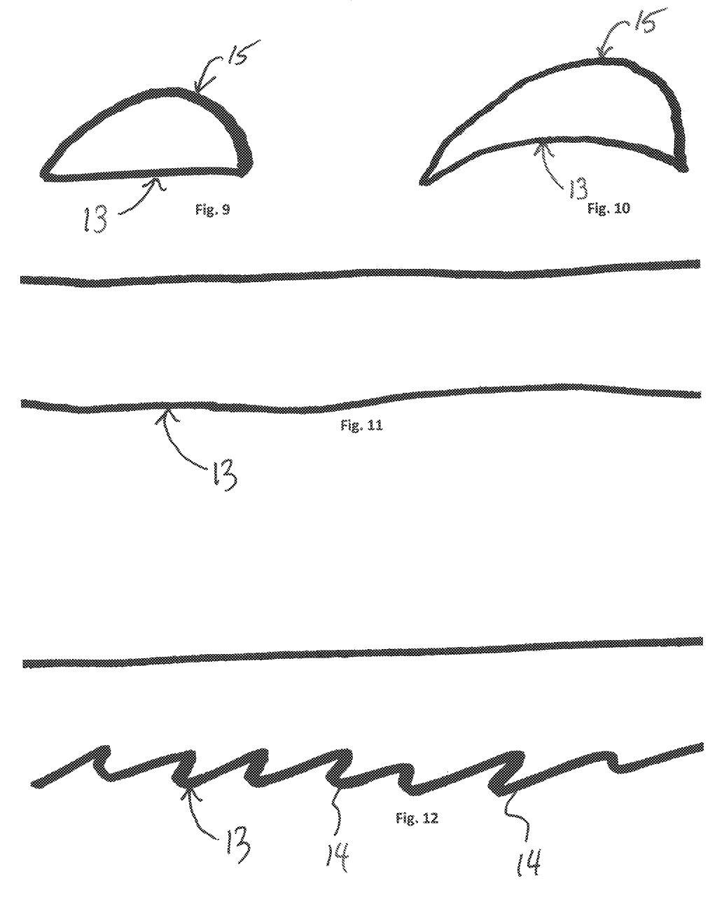

FIGS. 9 and 10 show a cross section of a medial portion 7 of a tension strip 1. In particular, in FIG. 9 the bottom surface 13 has a generally flat profile, the surface could be smooth, have ribs, or otherwise have a textured surface that is complementary to the bottom portion 12 of the tensions strip head 10. FIG. 10 illustrates another preferred embodiment wherein the cross section of the tension strip 1 is curved in an arcuate profile. An arcuate profile could increase surface area contact between a tension strip medial portion 7 and bone fragment when compared to a similarly sized tension strip 1 with a flat surface, thus increasing the friction between the tension strip 1 and bone fragment. The more friction between the tensions strip 1 and the bone fragment the less likely that the tension strip 1 can slip in a longitudinal or radial direction.

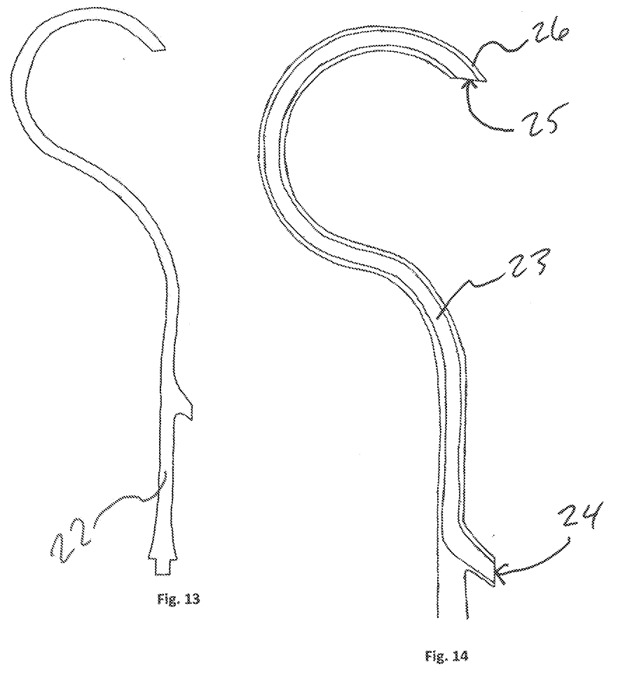

FIGS. 11 and 12 show the profile of a medial portion 7 of a tension strip 1. FIG. 11 shows a relatively smooth profile to reduce friction with tissue. FIG. 12 shows textured teeth 14 on the bottom surface 13 of the tension strip 1. The textured teeth 14 need not cover the entire length of the tension strip 1, the textured teeth 14 can be preferably tailored to cover a certain bone fragment diameter from 2 millimeter to 10 millimeter for small diameter bones to 10 millimeter to 100 millimeter diameter for larger bones, so called engagement portion since it engages the tension strip head 10. The bottom surface 13 of the tension strip 1 could have a textured surface for interfacing with the bone fragments and a different surface texture (such as ribs) for engaging the tensions strip head 10. The proximal portion 6 of the tensions strip 1 can be quite long relative the diameter of the target bone to enable the technician to easily handle the tensions strip 1 and engage the tension strip head 10 into the tensioning device 50. Thus, the tension strip 1 can have differentially textured bottom surfaces to improve engagement with bone and the tension strip head 10.

FIGS. 9 and 10 also illustrate a curved top side 15 of the tension strip 1. This curved shape can reduce interference with tissue surrounding the bone. Although tension strips 1 could be made of surgical grade metals, in a preferred embodiment the tension strip 1 can be manufactured of a bioresorbable material. Bioresorption rate can be increased by adjusting the surface area to volume ratio of the tension strip profile. Thus, bones that require a longer time to heal could have a thicker cross section or a rectangular cross section. Smaller bones or bones that do not require a long time to heal, i.e. less than six weeks can utilize tension strips 1 with a small cross section or a cross section that promotes quick bioresorption. In a preferred embodiment, a bioresorbable polymer is doped with a radiopaque material to allow patient monitoring via x-ray or fluoroscopic methods. Typical bioresorbable polymers include polylactic and polyglycolic acids, polyetheylene and polydioxanone. Other bioresorbable polymers are well known for different applications such as bioresorbable sutures. Radiopaque dopents are known in the industry, for example compounds containing iodine, barium sulfate and bismuth oxides. Other common biocompatible materials are PEEK polymer. Additionally, the material used for the tension strip 1 must be capable of being manufactured in a sterile environment or capable of post manufacture sterilization, commonly ethylene oxide, autoclave or irradiation. Tension strips must be capable of maintaining a range of pressure from 30-200 psi (207-1379 kPa) because the preferred pressure setting long bones is 100-150 psi (690-1,034 kPa).

Additionally, the tension strip 1 of a preferred embodiment is low profile and the guide member 22 has a similar low profile that reduces injury to tissue adjacent the bone fragment. The guide member conduit 23 is adapted to receive low profile tension strip 1 and reduce the overall profile of the guide member 22.

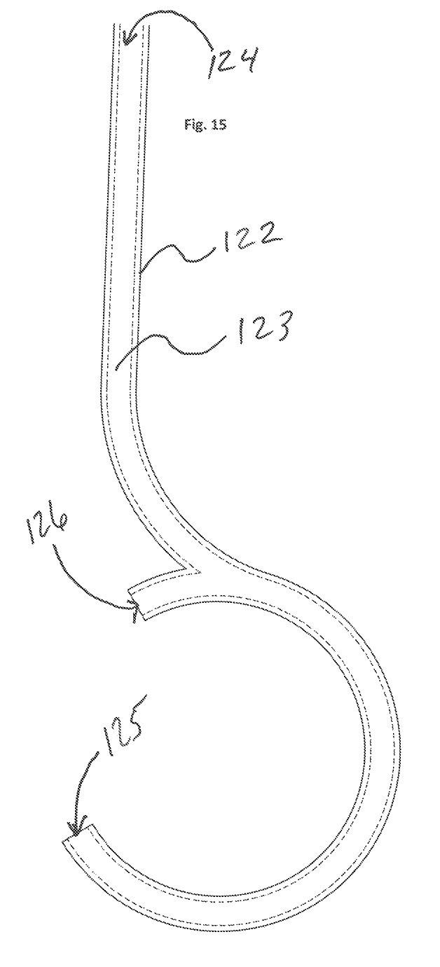

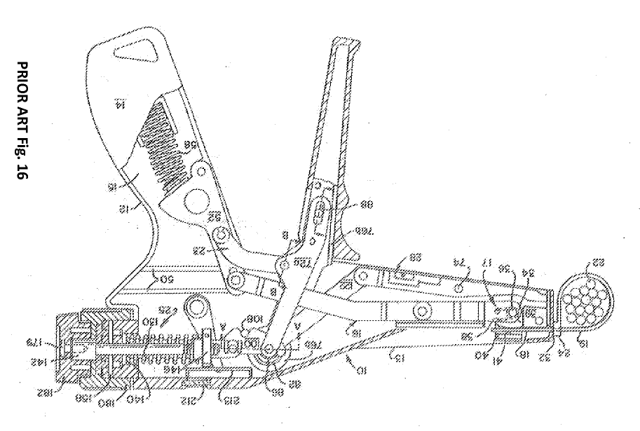

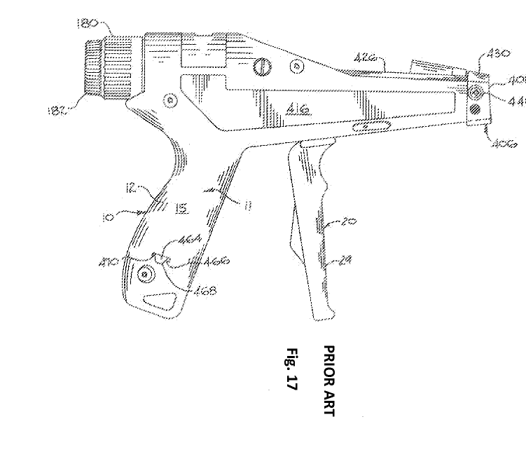

FIGS. 13 and 14 show an embodiment of a tensioning strip guide member 22. The guide member 22 is an elongate member, which is generally hook shaped with a conduit 23 for receiving a tensioning strip 1. A tension strip 1 is inserted into the conduit 23 with the head 10 of the tension strip 1 adjacent the distal opening 25 of the guide member 22. When a tension strip 1 is placed in this manner, the tension strip head 10 has a profile that is just large enough to match the profile of the guide member 22. Additionally, the tension strip proximal portion 6 may have a lower profile than the tension strip head 10 and the tension strip proximal portion 6 can fit in the narrow conduit 23 of the tension strip guide member 22. The distal end 26 of the guide member 22 is inserted through tissue and around a bone fragment. In this embodiment, the low profile tension strip head 10 allows tension strip guide member 22 to pass through tissue without snagging the tissue. This embodiment will reduce trauma to the tissue surrounding the bone. The tension strip guide member 22 is then removed while the technician holds the tension strip head 10 in place. Once the guide member 22 is completely removed the tension strip 1 is in position and the technician can attach the tension strip head 10 to a tensioning device 50 (FIGS. 16-19). The methods of attaching the tension strip 1 to the tension device 50 and actuating the tension device is more fully described below. In an alternative embodiment the conduit 23 of the tension strip guide member 22 is large enough to allow passage of a tension strip head 10 from a proximal opening 24 through a distal opening 25. FIG. 14 shows a distal portion 26 that has an angled cut so that it may lead and penetrate tissue with or without a tension strip 1 inserted into the conduit 23. Contrast FIG. 15, the distal opening 125 is blunt and the use of a tension strip 1 would aid in the penetration of tissue.

Preferred method of placement of a tension strip (FIG. 15):
1. Technician places tension strip proximal portion 6 into guide member distal opening 125 and pushes tension strip 1 into guide member conduit 123, the tension strip 1 may optionally protrude out of guide member medial opening 126 or remain in the conduit 123, the tension strip head 10 is in contact with distal opening 125 and occluding the guide member conduit 123.
2. The distal opening 125 of the loaded guide member 122 is then placed through the tissue around the bone.
3. Technician then grabs the tension strip head 10, holds tension strip head 10 in place and removes the guide member 122.
4. The tension strip 1 is then ready to attach to the tension device 50.

In this embodiment the guide member 122 would have an extremely low profile, just thick enough to accommodate the thin proximal portion 6 of the tension strip 1. Also, the low profile head 10 would actually act as an obturator to block the distal opening 125 from snagging tissue. The tension strip guide member 122 can be made of very strong metal such as aluminum or surgical steel so that the guide member 122 is strong enough to push though tissue and maintain the open conduit shape.

Alternatively, a method utilizes a larger profile guide member 122 and concomitantly larger conduit that could accommodate the tension strip 1 and tension strip head 10. In such a case the guide member 122 would be placed (with or without a tensions strip loaded) first, then multiple tension strips 1 could be fed through medial opening 126 to distal opening 125 as the guide member 122 is moved along the bone. This method would allow rapid placement of multiple tension strips 1.

In an alternative embodiment a tension strip 1 could be pushed through proximal opening 124 to avoid large tissue mass in unusual situations and then positioned to exit medial opening 126 or distal opening 125.

The guide member 22,122 disclosed in FIGS. 13-15 have conduits 23, 123 respectively, the conduit profile is intended to be complementary to the profile of the tension strip, such that the typical profile is flat thin rectangular shape. Alternatively, the conduit profile could accommodate tension strip 1 profiles as shown in FIGS. 9-10. By matching the conduit profile and the tension strip profile the overall profile can be minimized and reduce injury to tissue around the bone.

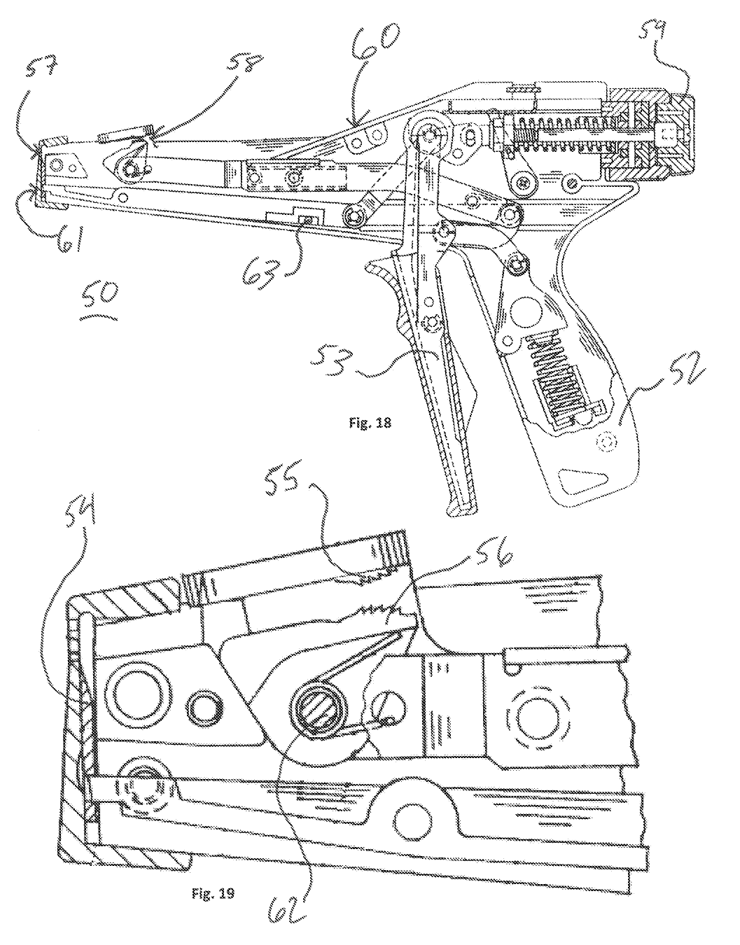

FIGS. 16-17 illustrate prior art and a preferred embodiment FIGS. 18-19, of a tensioning device 50. Tensioning devices are know in various industries with various combinations of properties for various field applications. The tensioning device 50 of the present invention comprises a handle 52 that a user holds to control positioning of tensioning device 50. The tensioning device 50 further comprises a lever 53 that when squeezed towards the handle 52 operates a ratcheting mechanism. The tensioning device 50 further comprises an elongate barrel 60 with a tensioning dial 59 at a proximal end and a blade 56 at a distal end. The distal end of the elongate barrel 60 has an entry channel 57 for receiving a tension strip proximal portion 6. Once a tension strip 1 is in place around a bone the head 10 is abutted against distal surface 61 of barrel 60 and a user feeds the proximal potion 6 of the tension strip 1 through the head 10 and into entry channel 57. The user can feed as much excess proximal portion 6 through the entry channel 57 until the proximal portion 6 exits through the top of the elongate barrel 60 via an exit channel 58. before the user needs to operate the ratcheting mechanism. The ratcheting mechanism has an upper grasper 55 and a lower grasper 56 that are actuated by lever 53 by pivoting about axis 62. FIG. 19 shows the upper grasper 55 and lower grasper 56 in the relaxed position (prior to applying pressure to the lever) and FIG. 18 shows the upper grasper 55 and lower grasper 56 in the actuated position (subsequent to applying pressure to the lever). The disclosed ratcheting mechanism shows that the proximal portion 6 has a textured surface on the top and bottom, alternative designs would only require one side of the proximal portion 6 to have texture and the graspers 55,56 are optional such that only one grasper is required to engage and pull a tension strip 1 through exit channel 58. The ratcheting mechanism allows the operator to maintain very accurate control, while there is virtually no tension applied to the tension strip 1 the ratcheting mechanism can pull ten to thirty millimeters length. As tension in the tension strip 1 builds the user can operate the lever 53 to move advance the tension strip 1 by less than one half millimeter per lever action.

The elongate barrel 60 further comprises a tension dial 59 at the proximal end and an on/off cutting switch 63. The tension dial 59 rotates to set the pressure at which tensioning stops. The on/off cutting switch 63 is shown in FIG. 18 is in the “on” position such that when pressure in the tension strip 1 reaches a predetermined pressure on the tension dial 59 a blade 54 cuts the proximal portion 6 adjacent the head 10. By placing the blade adjacent the head 10 less than one millimeter of excess material is retained on the tension strip 1. If the on/off switch 53 is moved to the forward position (not shown) the blade 54 will not automatically cut the tension strip 1, thus the user can make adjustments to placement of tension strips 1 and gradually add pressure optimally to the fractured bone.

In this inventive application it is critical that the tension strip 1 be cut as close to the tension strip head 10 as possible to reduce the total amount of material left in the patient as possible. However, there is occasion when the cutting of excess material should be done immediately and occasion to delay cutting the excess material. For example, in applications that require only one tension strip 1 it is time saving to set the tensioning device 50 to automatically cut the excess material. In other applications, for example, in fixing long bones, the tensioning may be done with several tension strips 1 and the user may want to place bone fragments under a slight tension to aid in alignment then come back to the tension strips 1 and increase the tension to a clinically beneficial pressure. By analogy, a mechanic tensions a wheel to an axle by tensioning the bolts in a criss cross fashion. Similarly, a user can adjust the tension on several tension strips 1 then comeback to each tension strip 1 and cut the excess material. So, in a preferred embodiment the functionality to turn on and turn off the automatic cutting function is desirable.

Another desirable feature of the preferred embodiment is the ability of the user to preselect what force to use to tension the bone fragment. It is well known in the industry that children's bone fractures depending on the bone age and cause of the fracture may require higher or lower compression to promote healing. Also, normal adult bone compression may be preselected to quickly set the bone. Additional care may be required or a lower compression force used in geriatric patients.

Another feature that has not been addressed in the prior art is the user ergonomics. In a preferred embodiment, FIG. 18, the elongate barrel 60 of the tensioning device 50 is long and narrow to improve user visualization of the target bone. Also, the tension strip 1 is fed through the tension device 50 such that the proximal portion 6 of the tension strip exits the tension strip head 10 channel through the entry channel 57 and out the top of the tensioning device 50 via the exit channel 58. This allows the user to get close to the bone and not have the tension strip 1 poke down and into the patient body or tissue. Also, due to the close positioning of the tension strip head 10 to the site of removing excess tension strip 1 the overall placement of the tension strip 1 will achieve a low profile. The low profile and reduced excess material will reduce the possibility of tissue damage or a user accidentally snagging excess tension strip.

It will be understood that various modifications can be made to the various embodiments of the present invention herein disclosed without departing from the spirit and scope thereof. For example, various devices are contemplated as well as various types of construction materials. Also, various modifications may be made in the configuration of the parts and their interaction. Therefore, the above description should not be construed as limiting the invention, but merely as an exemplification of preferred embodiments thereof. Those of skill in the art will envision other modifications within the scope and spirit of the present invention as defined by the claims appended hereto.

I claim:

1. A tension strip comprising a proximal portion a medial portion and a distal portion that is connected to a low profile head and the proximal portion passes through the head in only one direction, the medial portion is textured.

2. A tension strip placement method wherein a tension strip with a proximal portion, a medial portion and a distal portion connected to a low profile head is loaded into a guide member conduit at a distal opening and the low profile head occludes the distal opening.

3. A bone fixation device comprising a handle, an elongate barrel, the elongate barrel attached to a handle and a lever for creating pressure on a tension strip when the lever is squeezed, the lever actuates a ratcheting mechanism that engages a tension strip proximal portion with at least one grasper that pulls the tension strip proximal portion out an exit channel, the elongate barrel further comprising a blade for cutting the tension strip proximal portion.

4. The bone fixation device of claim 3 wherein the elongate barrel further comprises a tensioning dial that pre-sets a pressure to actuate the blade to automatically cut the tension strip proximal portion when the pre-set pressure on the tension strip is reached.

5. A bone fixation system comprising a tension strip and a guide member.
